(19) United States
(12) Patent Application Publication
(10) Pub. No.: US 2021/0275058 A1
(43) Pub. Date: Sep. 9, 2021

(54) SYSTEMS AND METHODS FOR AUTOMATED LOCALIZATION OF WEARABLE CARDIAC MONITORING SYSTEMS AND SENSOR POSITION-INDEPENDENT HEMODYNAMIC INFERENCE

(71) Applicant: Georgia Tech Research Corporation, Atlanta, GA (US)

(72) Inventors: Jonathan Zia, Atlanta, GA (US); Omer Inan, Atlanta, GA (US); Jacob Kimball, Atlanta, GA (US); Christopher Rozell, Atlanta, GA (US)

(21) Appl. No.: 17/316,290

(22) Filed: May 10, 2021

Related U.S. Application Data
(63) Continuation-in-part of application No. 17/187,585, filed on Feb. 26, 2021.
Application No. 17/187,585 is a continuation of application No. 16/935,882, filed on Jul. 22, 2020, now abandoned.
(60) Provisional application No. 62/877,404, filed on Jul. 23, 2019.

(51) Int. Cl. A61B 5/11; A61B 5/00; A61B 5/06

(52) U.S. Cl. CPC A61B 5/1102; A61B 5/065; A61B 5/7267; A61B 5/7221

(57) ABSTRACT

The disclosed technology includes devices and methods for a wearable cardiac monitoring system to determine morphological variability and localization of the wearable cardiac monitoring system. The disclosed technology can include receiving seismocardiographic data, determining a classifier of the seismocardiographic data, determining a signal quality index of the seismocardiographic data, determining a quality of the seismocardiographic data based on the classifier and the signal quality index, and outputting an indication of the quality of the seismocardiographic data to a graphical user interface.

### CROSS-REFERENCE TO RELATED APPLICATIONS

This application is a continuation-in-part of U.S. patent application Ser. No. 17/187,585, filed 26 Feb. 2021, which is a continuation of U.S. patent application Ser. No. 16/935,882, filed 22 Jul. 2020, which claims the benefit of U.S. Provisional Patent Application No. 62/877,404 filed 23 Jul. 2019, each of which is hereby incorporated by reference as if fully set forth below.

### STATEMENT REGARDING GOVERNMENT SUPPORT

This invention was made with government support under Award No. 1R01HL134619-A1 awarded by the National Institutes of Health and UL1TR002378 awarded by the National Center for Advancing Translational Sciences. The government has certain rights in the invention.

### FIELD OF DISCLOSURE

The disclosed technology relates generally to wearable cardiac monitoring systems and, more particularly, to systems and methods for automated localization of wearable cardiac monitoring systems and generation of physiological inferences using wearable cardiac monitoring systems measuring precordial acceleration.

### BACKGROUND

Seismocardiography is a technique of measuring the movement of the chest wall in response to underlying cardiovascular events. Seismocardiography, for example, can be used to measure valvular events such as aortic opening (AO) and aortic closing (AC). Data collected by seismocardiography can be compiled into a seismocardiogram to enable medical professionals to make an inference of major prognostic factors of heart disease such as pre-ejection period (PEP), left ventricular ejection time (LVET), and pulse transit time (PTT).

By its nature, the morphology of a seismocardiographic (SCG) waveform is highly transient in the time domain, influenced by the coupling of the vascular system with the chest wall, the chest wall with the sensing system, and by the patient's physiological state. Consequently, morphological variability poses a significant challenge in processing SCG signals. Furthermore, when a seismocardiography signal is obtained by the sensing system, the information extracted from the obtained signal can vary significantly based on where the sensor is placed; thus, misplacement of the sensor can lead to readings that are not reliable. This is particularly a problem when a seismocardiogram is used during outpatient continuous monitoring because the patient typically is not trained to properly position the wearable sensor and because the wearable sensor can be knocked out of position while worn by the patient. These variations associated with obtaining and recording an SCG signal pose significant challenges for professionals to determine whether the obtained bio-signal, and any resultant seismocardiogram, is of sufficient quality to be used in informing patient care. Oftentimes, the challenges in properly positioning the wearable sensor render the sensor, and information it obtains, useless because the underlying processing system cannot properly account for the misplacement of the sensors.

What is needed, therefore, is a system and method that can determine a location of the sensor on the chest wall and correct distortions of extracted sensor information resulting from sensor misplacement or morphological variations, thus providing an accurate seismocardiogram based on the detected SCG signals.

### SUMMARY

These and other problems can be addressed by the technologies described herein. Examples of the present disclosure relate generally to systems and methods for automated morphological variability detection and localization of wearable cardiac monitoring systems measuring precordial acceleration.

The disclosed technology can include a method of detecting morphological changes using a wearable cardiac monitor. The method can include receiving seismocardiographic data from a motion sensor and determining, based at least in part on the seismocardiographic data, a classifier of the seismocardiographic data. The method can include determining, based at least in part on the seismocardiographic data, a signal quality index of the seismocardiographic data and determining, based on the classifier and the signal quality index, a quality of the seismocardiographic data. The method can further include outputting, to a graphical user interface, the quality of the seismocardiographic data.

The classifier can include multiple classifiers and the method can include performing ensemble prediction by utilizing the multiple classifiers to create an ensemble classifier. The method can also include retrieving a reference template and determining the signal quality index by determining a distance between the seismocardiographic data and the reference template. The reference template can be representative of seismocardiographic data.

The method can include estimating the distance between the seismocardiographic data and the reference template by dynamic-time feature matching. Dynamic-time feature matching can include determining a local minimum and a local maximum of the seismocardiographic data and determining a local minimum and a local maximum of the reference template. The method can include determining a prohibited intersection point, determining a candidate point, and generating a warp path through the candidate point.

The method can include retrieving a location-specific reference template and determining a location of the motion sensor by at least determining a distance between the seismocardiographic data and the location-specific reference template. The location-specific reference template can be representative seismocardiographic data at a predetermine location.

The method can include determining a hemodynamic factor by modeling the seismocardiographic data as a stochastic sample from an underlying dynamical system. The method can include performing a seismocardiogram generative factor encoding, comparing the seismocardiographic data to a low-dimensional subspace, and applying a position-specific regression to the low-dimensional subspace to determine a hemodynamic factor.

The method can include comparing the seismocardiographic data to a low-dimensional manifold, performing a manifold approximation to analyze a distance traveled along the low-dimensional manifold, and determining a hemodynamic change based on the distance traveled along the low-dimensional manifold.

The disclosed technology can include a wearable cardiac monitoring system that can include a controller and a motion sensor configured to detect movement of a user's chest and output seismocardiographic data to the controller. The controller can include a non-transitory, computer-readable medium having instructions stored thereon that, when executed by one or more processors, cause a controller to receive the seismocardiographic data from the motion sensor and determine, based at least in part on the seismocardiographic data, a classifier of the seismocardiographic data. The instructions can cause the controller to determine, based at least in part on the seismocardiographic data, a signal quality index of the seismocardiographic data and determine, based on the classifier and the signal quality index, a quality of the seismocardiographic data. The instructions can cause the controller to output, to a graphical user interface, an indication of the quality of the seismocardiographic data.

The classifier can comprise a property of a source distribution of the seismocardiographic data. The classifier can comprise multiple classifiers and the instructions, when executed by the one or more processors, can cause the controller to perform ensemble prediction by utilizing the multiple classifiers to create an ensemble classifier.

The instructions can further cause the controller to retrieve a reference template and determine the signal quality index by determining a distance between the seismocardiographic data and the reference template. The reference template can be representative of seismocardiographic data. The instructions can further cause the controller to estimate the distance between the seismocardiographic data and the reference template by dynamic-time feature matching. The dynamic-time feature matching can include determining a local minimum and a local maximum of the seismocardiographic data and determining a local minimum and a local maximum of the reference template. Dynamic-time feature matching can include determining a prohibited intersection point, determining a candidate point, and generating a warp path through the candidate point.

The instructions can further cause the controller to retrieve a location-specific reference template and determine a location of the motion sensor by at least determining a distance between the seismocardiographic data and the location-specific reference template. The location-specific reference template can be representative seismocardiographic data at a predetermine location.

The instructions can cause the controller to perform a seismocardiogram generative factor encoding, compare the seismocardiographic data to a low-dimensional subspace, and apply a position-specific regression to the low-dimensional subspace to determine a hemodynamic factor.

The instructions can further cause the controller to compare the seismocardiographic data to a low-dimensional manifold, perform a manifold approximation to analyze a distance traveled along the low-dimensional manifold, and determine a hemodynamic change based on the distance traveled along the low-dimensional manifold.

The controller can be configured to wirelessly receive the seismocardiographic data from the motion sensor. The controller can be a remote server configured to wirelessly receive the seismocardiographic data from the motion sensor. The controller can be integrated with the motion sensor into a single wearable device.

The instructions can cause the controller to receive a first input to change a threshold value of the classifier and receive a second input to change a threshold value of the signal quality index.

Additional features, functionalities, and applications of the disclosed technology are discussed herein in more detail.

### BRIEF DESCRIPTION OF THE FIGURES

The accompanying drawings, which are incorporated in and constitute a part of this specification, illustrate various aspects of the presently disclosed subject matter and serve to explain the principles of the presently disclosed subject matter. The drawings are not intended to limit the scope of the presently disclosed subject matter in any manner.

FIG. 1 is an illustration of a wearable cardiac monitoring system, in accordance with the disclosed technology.

FIG. 2 is an illustration of a method of analyzing seismocardiogram signals, in accordance with the disclosed technology.

FIG. 3 is graph of analyzed seismocardiogram signals, in accordance with the disclosed technology.

FIG. 4 is an illustration of a method of analyzing seismocardiogram signals, in accordance with the disclosed technology.

FIG. 5A is an illustration of various sensor locations, in accordance with the disclosed technology.

FIG. 5B is an illustration of a method of analyzing seismocardiogram signals and determining a location of a sensor, in accordance with the disclosed technology.

FIG. 6 is an illustration of a method of analyzing seismocardiogram signals, in accordance with the disclosed technology.

FIG. 7 is an illustration of a method of analyzing seismocardiogram signals, in accordance with the disclosed technology.

FIG. 8 illustrates various graphs of seismocardiogram signals in accordance with the disclosed technology.

FIGS. 9A-9C illustrates various graphs of seismocardiogram signals in accordance with the disclosed technology.

FIG. 10 is an illustration of a method of analyzing seismocardiogram signals, in accordance with the disclosed technology.

FIGS. 11A-11E illustrates various graphs of seismocardiogram signals in accordance with the disclosed technology.

### DETAILED DESCRIPTION

The disclosed technology relates to improved systems and methods for automated morphological variability detection and localization of wearable cardiac monitoring systems measuring precordial acceleration. The disclosed technology can include wearable cardiac monitors (e.g., holter monitors and other wearable devices that capture cardiovascular data such as electrocardiograph (ECG)) that incorporate sensors used to capture signals that can be processed to create seismocardiograms and the underlying processing systems to both (1) localize the sensor and (2) correct for sensor misplacement and morphological variability when inferring AO and AC event timing from the signal. The disclosed technology can greatly improve the user experience when using the wearable cardiac monitors because the user is not required to place the sensor in a single specific location on his or her chest. Furthermore, the disclosed technology includes methods for extracting bio-signal data independently of waveform morphology to yield more consistent data when applied to a heterogeneous population, which is essential in ambulatory and outpatient environments.

As will become apparent throughout this disclosure, the disclosed technology can include systems and methods for improving sensor localization by using, among other things, a variant of dynamic time warping (DTW) called dynamic time feature matching (DTFM) to infer sensor location from seismocardiography signals using ensembled quadratic discriminant analysis (QDA) classification. Furthermore, the disclosed technology can include dynamic systems modeling used to correct estimated AO and AC values based on the location of the sensor once the sensor has been localized. Combining these methods can yield a system which both localizes the wearable sensor on the chest wall and corrects distortions of extracted sensor information resulting from sensor misplacement. In other words, the disclosed technology can enable position-specific mapping of seismocardiographic data to infer hemodynamic variables. By localizing the sensor, the disclosed technology can be used either to inform the patient where to properly place the sensor on his or her chest or to automatically compensate for the sensor misplacement without requiring the sensor to be relocated.

Although various aspects of the disclosed technology are explained in detail herein, it is to be understood that other aspects of the disclosed technology are contemplated. Accordingly, it is not intended that the disclosed technology is limited in its scope to the details of construction and arrangement of components expressly set forth in the following description or illustrated in the drawings. The disclosed technology can be implemented and practiced or carried out in various ways. In particular, the presently disclosed subject matter is described in the context of being systems and methods for automated localization and misplacement correction of wearable cardiac monitoring systems measuring precordial acceleration. The present disclosure, however, is not so limited, and can be applicable in other contexts. Accordingly, when the present disclosure is described in the context of automated localization and misplacement correction of wearable cardiac monitoring systems measuring precordial acceleration, it will be understood that other implementations can take the place of those referred to.

It should also be noted that, as used in the specification and the appended claims, the singular forms “a,” “an,” and “the” include plural references unless the context clearly dictates otherwise. References to a composition containing “a” constituent is intended to include other constituents in addition to the one named.

Also, in describing the disclosed technology, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, the disclosed technology can include from the one particular value and/or to the other particular value. Further, ranges described as being between a first value and a second value are inclusive of the first and second values. Likewise, ranges described as being from a first value and to a second value are inclusive of the first and second values.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

It is also to be understood that the mention of one or more method steps does not preclude the presence of additional method steps or intervening method steps between those steps expressly identified. Moreover, although the term “step” can be used herein to connote different aspects of methods employed, the term should not be interpreted as implying any particular order among or between various steps herein disclosed unless and except when the order of individual steps is explicitly required. Further, the disclosed technology does not necessarily require all steps included in the methods and processes described herein. That is, the disclosed technology includes methods that omit one or more steps expressly discussed with respect to the methods described herein.

The components described hereinafter as making up various elements of the disclosed technology are intended to be illustrative and not restrictive. Many suitable components that would perform the same or similar functions as the components described herein are intended to be embraced within the scope of the disclosed technology. Such other components not described herein can include, but are not limited to, similar components that are developed after development of the presently disclosed subject matter.

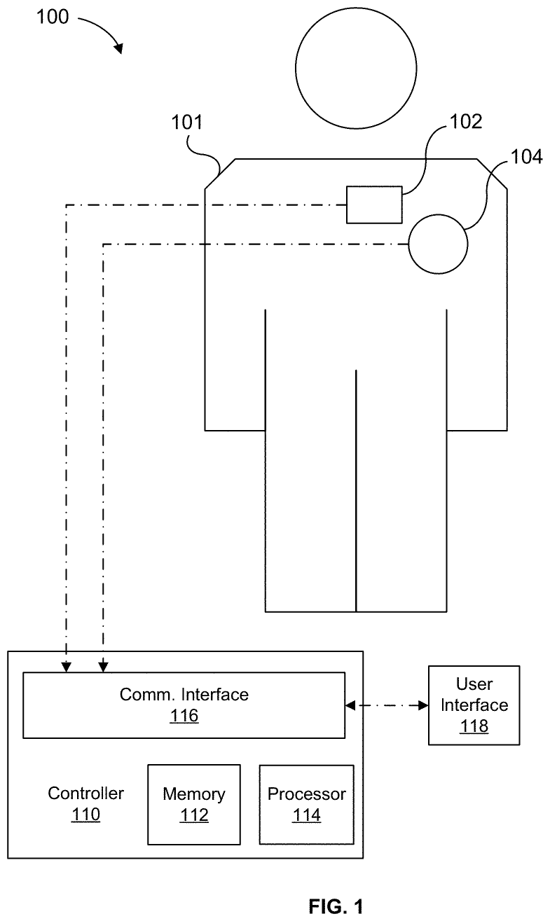

Referring now to the drawings, in which like numerals represent like elements, the present disclosure is herein described. FIG. 1 illustrates a system 100 for, among other things, automated morphological variability detection and localization of wearable cardiac monitoring systems measuring precordial acceleration. The system 100 can include a motion sensor 102 and an electrical activity sensor 104 that can be worn by, or otherwise placed on, a patient 101. The motion sensor 102 and the electrical activity sensor 104 can both be configured to output a detected signal to a controller 110. As will be described in greater detail herein, the controller 110 can be configured to receive data from the motion sensor 102 and the electrical activity sensor 104, analyze the received data, and output data to a user interface 118 so that the patient 101, doctor, nurse, technician, and/or any other medical professional is able to make decisions about the patient's 101 health.

The motion sensor 102 can be any type of sensor configured to detect movement of the patient's 101 chest or body caused by underlying cardiovascular events and output motion signals (referred to herein as a seismocardiographic (SCG) signal) indicative of the detected movement. The motion sensor 102, for example, can be an accelerometer, a three-axis accelerometer, a gyroscope (or gyrometer), an inertial measurement unit (IMU), or any other suitable type of sensor that can detect movement of the patient's 101 chest or body. The electrical activity sensor 104 can be any type of sensor configured to detect electrical activity of the patient's 101 body and output electrical activity signals that can be used to produce an electrocardiogram (ECG) and/or an impedance cardiogram (ICG). The electrical activity sensor 104 can include electrodes placed on the skin of the patient 101 that are capable of detecting electrical changes that are produced by the patient's 101 heart.

Although depicted as being two separate sensors, the motion sensor 102 and the electrical activity sensor 104 can both be mounted into a single unit such that the motion sensor 102 and the electrical activity sensor 104 comprise a single device. Furthermore, although depicted as being in communication with the controller 110 that is separate from the patient 101, the controller 110 can be integrated with the motion sensor 102 and the electrical activity sensor 104 such that the controller 110, the motion sensor 102, and the electrical activity sensor 104 comprise a single unit. Alternatively, portions of the controller 110 can be integrated with the motion sensor 102 and/or the electrical activity sensor 104 while other portions of the controller 110 can be separated from the motion sensor 102 and the electrical activity sensor 104. For example, a memory 112 and a processor 114 of the controller 110 can be integrated with the motion sensor 102 and the electrical activity sensor 104 to perform some of the actions described herein while further actions are performed by the controller 110 that is remote from the motion sensor 102 and the electrical activity sensor 104. If the controller 110, or portions of the controller 110 are separated from the motion sensor 102 and the electrical activity sensor 104, the motion sensor 102 and the electrical activity sensor 104 can either be in wireless communication or wired communication with the controller 110.

The controller 110 can have a memory 112, a processor 114, and a communication interface 116. The controller 110 can be a computing device (e.g., a desktop, a laptop, a tablet, a mobile device, a remote server, or any other suitable type of computing device) configured to receive biometric data from the motion sensor 102 the electrical activity sensor 104. One of skill in the art will appreciate that the controller 110 can be installed in any location, provided the controller 110 is in communication with at least some of the components of the system 100 (including the motion sensor 102 and the electrical activity sensor 104). Furthermore, the controller 110 can be configured to send and receive wireless or wired signals and the signals can be analog or digital signals. The wireless signals can include Bluetooth™, BLE, WiFi™, ZigBee™, infrared, microwave radio, or any other type of wireless communication as may be suitable for the particular application. The hard-wired signal can include any directly wired connection between the controller and the other components. The digital connection can include a connection such as an Ethernet or a serial connection and can utilize any suitable communication protocol for the application such as Modbus, fieldbus, PROFIBUS, SafetyBus p, Ethernet/IP, or any other suitable communication protocol for the application. Furthermore, the controller 110 can utilize a combination of wireless, hard-wired, and analog or digital communication signals to communicate with and control the various components. One of skill in the art will appreciate that the above configurations are given merely as non-limiting examples and the actual configuration can vary depending on the particular application.

The controller 110 can include a memory 112 that can store a program and/or instructions associated with the functions and methods described herein and can include one or more processors 114 configured to execute the program and/or instructions. The memory 112 can include one or more suitable types of memory (e.g., volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like) for storing files including the operating system, application programs (including, for example, a web browser application, a widget or gadget engine, and or other applications, as necessary), executable instructions and data. One, some, or all of the processing techniques or methods described herein can be implemented as a combination of executable instructions and data within the memory.

The controller 110 can also have a communication interface 116 for sending and receiving communication signals between the various components. Communication interface 116 can include hardware, firmware, and/or software that allows the processor(s) 114 to communicate with the other components via wired or wireless networks, whether local or wide area, private or public, as known in the art. Communication interface 116 can also provide access to a cellular network, the Internet, a local area network, or another wide-area network as suitable for the particular application.

Additionally, the controller 110 can have or be in communication with a user interface 138 for displaying system information and receiving inputs from a user. The user interface 138 can be installed locally on the system 100 or be a remotely controlled device such as a mobile device. The user, for example, can input data to set one or more thresholds used by the controller 110 to determine actions based on system 100 conditions.

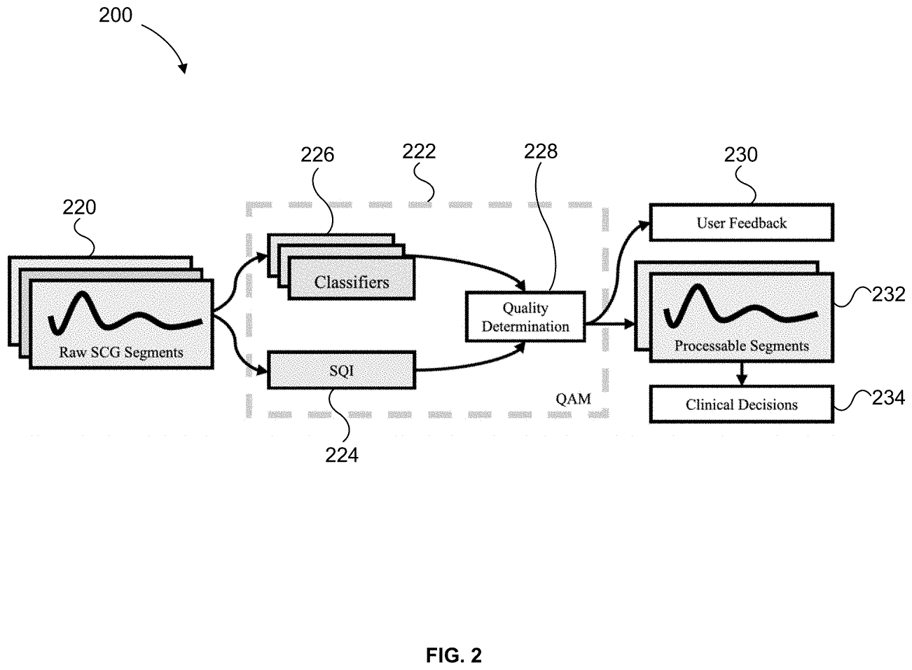

FIG. 2 illustrates a method 200 of determining a quality of an SCG signal as detected by at least the motion sensor 102. As illustrated in FIG. 2, the method can include receiving SCG signal segments 220 and performing a quality assessment on the received SCG signal segments 220 at a quality assessment module 222. The SCG signal segments 220 can include motion signals generated by the sensor 102 in response to detecting movement of the patient's 101 chest resulting from both precordial acceleration and the patient's 101 movement. The SCG signal segments 220 can be processed by a bandpass filter (e.g., 1-40 Hz) and/or some other blind source separation to help isolate the data indicative of precordial acceleration from the data indicative of movement of the patient's 101 chest. The quality assessment module 222, as will be explained in greater detail herein, can be configured to determine a signal quality index (SQI) 224 and classifiers 226 of the received signal segments 220 to generate a quality determination 228. The information generated as part of the quality determination 228 can then be used by a medical professional to determine whether the received SCG data (i.e., the SCG signal segments 220) are of sufficient quality to make determinations of the patient's 101 health. As further show in in FIG. 2, the method 200 can include generating user feedback 230 based on the quality determination 228, and outputting that feedback to provide the user with information about the signal quality. For example, the feedback 230 can be outputted for display on the user's computing device or a display of the system 100, and the feedback 230 can include information indicating whether the motion sensor 102 and/or electrical activity sensor 104 is properly positioned, whether the signal is of sufficient quality to use for making informed health decisions, and information about how the user can obtain a signal of higher quality. If the quality determination 228 indicates that the signal quality is sufficient, the method 200 can further include analyzing processable segments 232 of the SCG signal to generate a seismocardiograph and outputting the seismocardiograph data for clinical decisions 234.

As noted and as shown in FIG. 2, an aspect of the quality assessment module 222 can include generating an SQI 224 of the SCG signal. The SQI 224 can be a function of the inverse distance between the SCG signal and a diverse set of reference templates. As the performance of the SQI 224 is dependent on the type of distance metric used to calculate the inverse distance between the SCG signal and a diverse set of reference templates, the disclosed technology can include dynamic-time feature matching (DTFM) as a method of distance estimation as will be described in greater detail herein.

As will be appreciated by one of skill in the art, existing methods of biosignal distance estimation, including dynamic time warping (DTW), are limited in their application. Implicit in the mapping generated by DTW are assumptions about time-series feature correspondence—namely, local minima and maxima. Since SCG waveforms are highly prone to motion-artifact noise, the assumptions made by DTW to minimize the Euclidean distance can distort the true relationships between these features and ultimately underestimate the true distance between the signals. To correct this limitation, the disclosed technology can determine that any valid mapping between an SCG signal and a template will necessarily match each feature in the template to a corresponding feature in the signal whenever possible. Because seismocardiography is highly prone to motion artifacts in ambulant subjects, the number of features in the signal will exceed the template in the vast majority of cases. In other words, because the motion sensor 102 is prone to detect motion artifacts caused by patient movement that are unrelated to cardiovascular events, the disclosed technology can be used to match features of the SCG signal to features of a template to interpret the received SCG signal data and generate a clearer seismocardiograph.

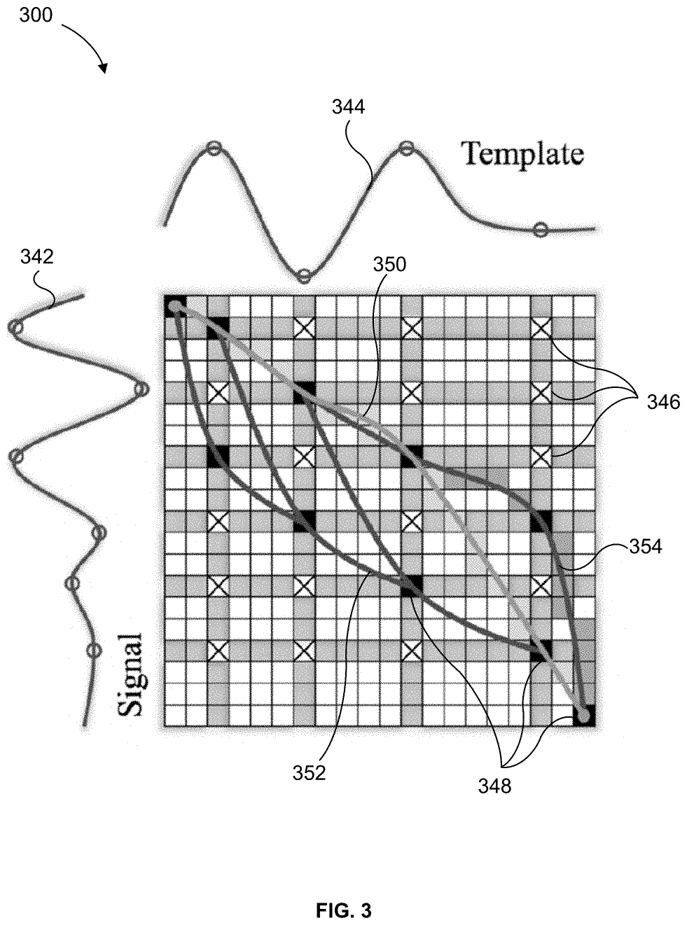

FIG. 3 illustrates a graph of distance estimation using the disclosed DTFM method 300. As noted, the disclosed DTFM method 300 can impose additional constraints on the warp path to generate a valid warp path of the SCG signal. In certain embodiments, the DTFM method 300 can include identifying all local minima and maxima in the SCG signal 342 (such as SCG segments 220) and a reference template 344. The reference template 344 can be a template that is indicative of pre-recorded SCG signals having known characteristics. For example, the template 344 can be a recorded SCG signal where the location of the sensor 102 is known or an SCG signal where an underlying cardiovascular condition is known. As will be appreciated, these reference templates 344 can be pre-recorded and stored in the memory 112 of the controller 110. Alternatively, or in addition, references templates 344 that are user-specific may be generated and stored in the memory 112. For example, the patient 101 may be prompted or instructed to place the sensor 102 in different locations on his or her chest and one or more reference templates 344 can be generated for each position of the sensor 102 on the patient's chest. As depicted in FIG. 3, the rows and columns of the warping matrix corresponding to timestamps of the signal 342 and template 344 features respectively are aligned with the signal 342 and the template 344. Points lying at the intersection of these rows and columns are referred to as “intersection” points; passing the warp path through intersection points results in matching a feature in the signal 342 to a feature in the template 344.

The DTFM method 300 can include identifying prohibited intersection points 346 in the warping matrix, as shown with crossed squares in FIG. 3. Identifying prohibited intersection points 346 can include determining that passing the warping path through a particular point would result in either (1) matching a local minimum to a local maximum or (2) stretching the feature in the signal or template beyond some pre-defined limit. The DTFM method 300 can include removing all prohibited points 346 and denoting all remaining intersection points as “candidate” points 348, as indicated by the solid boxes in FIG. 3. The DTFM method 300 matches all template 344 features with a signal 342 feature, if possible, to determine an optimal warp path 350. The DTFM method 300 can include repeating this process for each template 344 to determine the best valid warp path 350 based on the features of the template 344 and the signal 342. Thus, valid warp paths are those that pass through a candidate point 348 associated with each template 344 feature whenever it is possible to do so—namely, whenever (1) the feature has corresponding candidate points 348 and (2) doing so would not violate pre-existing constraints. The optimal path 350 can then be chosen as the valid path that minimizes the Euclidean distance between the warped signals.

Since there generally exists only one optimal warp path 350 between any two points in the matrix—as defined by minimizing Euclidean distance—the set of valid warp paths 352 can be small in number. Because the number of valid warp paths 352 is relatively small, the DTFM method 300 can rapidly and efficiently identify all valid warp paths 352 and determine the optimal warp path 350 based on the identified valid warp paths 352. To illustrate, in the example depicted in FIG. 3, multiple valid warp paths 352 are depicted while only a single optimal warp path 350 is depicted. The optimal warp path 350 is chosen in part because the third maximum in the signal 342 is determined to be aberrant, and the third minimum in the signal 342 is mapped to the second minimum in the template 344 resulting in the optimal warp path's 350 route as compared to a DTW warp path's 354 route.

As described above, the SQI 224 as illustrated in FIG. 2 is a function of the inverse distance between a captured signal (e.g., signal 342) and a reference template (e.g., template 344), shown in the following Equation (1).

$$SQI ( s , t ) {=}^{Δ} exp ( \frac{- λ D ( s , t )}{L ( s , t )} ) \qquad Equation ( 1 )$$

where s and t are the captured signal and reference template respectively, D(⋅) is the distance function, L is the length of the warped signal, and λ is an optional distance penalty. Unit SQI is achieved when there is no distance between the signals, and the SQI approaches zero exponentially as the sample-averaged distance increases. Since the length of signals (i.e., SCG signals 220) after warping may vary, L is intended to normalize the D by the length of the signal, yielding a distance-per-datapoint. λ determines the decay rate of the exponential term and thus only influences the range in [0, 1] in which scores commonly fall.

Because the reliability of the SQI 224 is highly dependent on the quality of the template, and because there is no reference standard for seismocardiography, using a single template may not yield a valid SQI 224 for all patients. Thus, the disclosed technology can include a set of templates T={t1, t2, . . . , t|T|}. The SQI 224 can therefore be defined over the template set T as

$$( s ) {=}^{Δ} SQI ( s , t ) \qquad Equation ( 2 )$$

where |T| is the number of elements in set T. By increasing the size and diversity of the template set, Equation (2) provides a more reliable SQI 224.

Using a population approach addresses the problem of determining the quality of the templates themselves. This task can be intractable both due to the lack of an objective reference standard and because template quality can vary situationally. As the number and diversity of templates increase, the influence of low-quality templates is averaged and thereby diminished, while representative templates can drive the overall SQI 224 higher or lower. As will be appreciated, this improves SQI 224 stability without incorporating subjective assumptions about template quality. Notably, scaling the score of each template by a static value—related to its quality, for instance—would not change SQI performance; though the final value can change, relative scores assigned to the signals would remain consistent, as all scores are equally affected by scalar weights.

To determine an appropriate classifier 226 for SCG signals, SCG signals drawn from a source distribution—or class—can generally result in a higher SQI from templates 344 created from the same class compared to others. If this assumption always held true, classification would be quite simple: given N classes, a template (i.e., template 344) set can be defined as T={t1, t2, . . . , t|T|} where tᵢ is a template created from signals from the ith class. Classifying the input signal s can be determined by

$$( s ) = {arg max}_{i} SQI ( s , {t}_{i} ) \qquad Equation ( 3 )$$

where C*_{T} (s) is the class of s predicted by template set T. The selected class is therefore the value of the argument i which maximizes the SQI 224. Since there is no known prototypical SCG signal, the classifier C*_{T} may not generalize well due to its high dependence on template quality.

The system 100 can be configured to compensate for template quality variability by determining the prior likelihood of the template to give a certain SQI 224 to signals from each class. To do this, the disclosed technology can re-frame the classification problem in the above Equation (3) as

$$( s ) = {arg max}_{i} {P}_{Y \vert X} ( Y = i \vert X = x ) \qquad Equation ( 4 )$$

P_{Y|X} is the probability that the true class of s is Y given the vector x of SQIs assigned to the signal by the template set.

Using Bayes' rule, Equation (4) is equivalent to

$$\begin{matrix} ( s ) = {arg max}_{i} \frac{{P}_{X \vert Y} ( X = x \vert Y = i ) {P}_{Y} ( Y = i )}{{P}_{X} ( X = x )} \\ = {arg max}_{.} {P}_{X \vert Y} ( X = x \vert Y = i )\end{matrix} \qquad Equation ( 5 )$$

since x is known and one of skill in the art can assume that all classes are equiprobable. The system 100 can learn the probability distribution based on training data, a technique called Bayes estimation. The learning process can be greatly accelerated by imposing assumptions on the probability distribution; among the most common types are linear discriminant analysis (LDA), in which the distributions are assumed to be Gaussian with fixed covariance across classes; and quadratic discriminant analysis (QDA), in which the covariance restriction is lifted.

Even with Bayesian methods, accurate classification with Equation (5) still relies on the quality of the template 344 set, and thus C_{T} can be a weak classifier. However, predictive performance of this method can improve by using ensemble prediction. As is understood by those of skill in the art, ensemble prediction is a robust technique whereby several weak classifiers act in unison to generate stronger predictions. As predictive performance of an ensemble classifier relies on diversity of its members, methods such as bagging and boosting can also be employed during training.

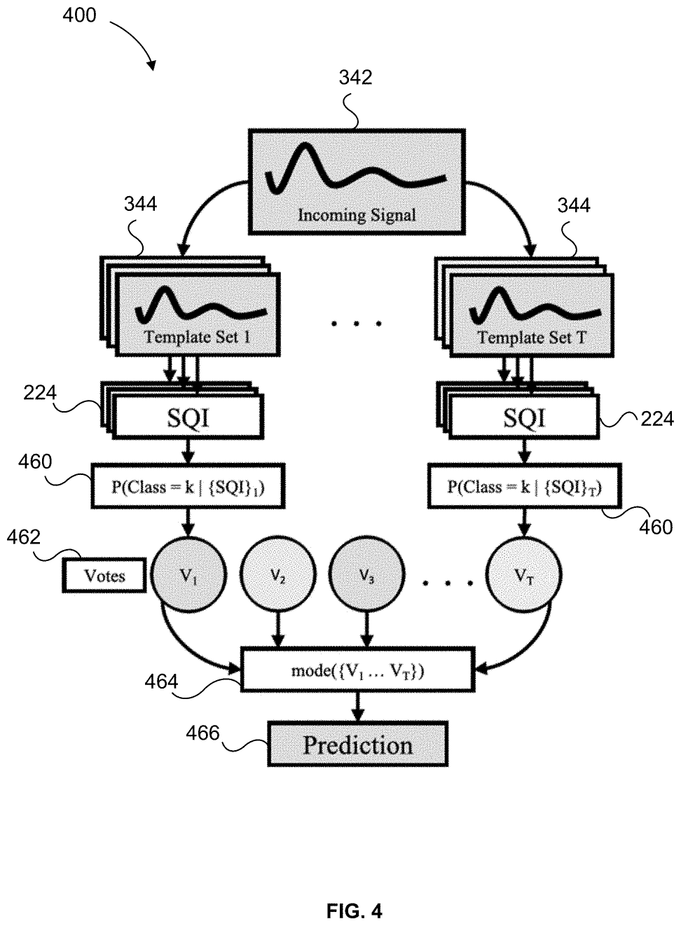

As illustrated in FIG. 4, the system 100 can be configured to apply ensemble prediction 400 to this method 300 and a superset M composed of unique template 344 sets can be used such that M={T1, T2, . . . , T|M|} where |M| is the number of template 344 sets. The method 400 can include training each classifier 460 as per Equation (5) and obtaining a majority vote 464 from the superset M of votes 462 on signal s as

V_{M}(s)= [symbol] [symbol] (s), Equation (6)

which returns the most common prediction 466 across all templates in set M. In other words, a template 344 set is composed of a group of templates, each derived from SCG signals belonging to a different class. Each template 344 can be used to assign the incoming signal an SQI 224. For each template 344 set, the method 400 can include mapping the SQI 224 values to the most likely classification (classifier 460) (accounting for variability in template 344 quality), which represents the vote 462 of the template 344 set. The system 100 then selects the prediction 466 that is the mode class (majority vote 464) of all template set votes 462.

As will be appreciated, by selecting diverse template 344 sets—such as by constructing each template 344 set from a different subject's data—the generalizability of this model improves, becoming less sensitive to the quality of any individual template 344. This property is valuable in SCG signal processing, as quality templates 344 are difficult to identify.

The ensemble prediction 400, as illustrated in FIG. 4, also can be used by the system 100 to detect and localize sensor (e.g., motion sensor 102 and/or electrical activity sensor 104) misplacement. For example, templates 344 can be generated and stored in the memory 112 of the controller 110 by obtaining SCG data when placing the sensor in various locations on the patient's chest. Each location can be denoted with its own individual identification (e.g., C (center), L (left), R (right), T (top), and B (bottom), etc.). Each template 344 generated at each location can have its own characteristics that can later be used to determine a location of the sensor based on the obtained SCG data.

To illustrate, using the five location indications for templates described above (i.e., C (center), L (left), R (right), T (top), and B (bottom)), multiple subject-specific templates 344 can be generated based on obtained SCG data. Each template 344 set can be composed of five templates 344, generated from the subject's resting-period segments from each of the five accelerometer positions. Thus, the template set for each subject S can be T_{S}={t^{s}_{C}, t^{s}_{L}, t^{s}_{R}, t^{s}_{T}, t^{s}_{B}}. These template sets can then combined into a superset M={T1, T2, . . . , T10}. A new superset M_{S} can then be defined based on selected subjects such that M_{S}={Ti}, i∈[1, 10], i≠S. To use this superset for classification purposes, the prior distributions of selected subjects can be characterized to obtain C_{T} as described above. This setup can be analogous to treating each set as a separate QDA classifier.

For each set Tᵢ in MS, these parameters can be learned by obtaining the SQI 224 for each template 344 tⁱⱼ, j∈{C, L, R, T, B}. μi can then be calculated as the mean score of each template 344 t^{j}ⱼ for each class 460. Each element in Σi can be calculated as the covariance of SQIs 224 between the five templates in Tᵢ for each class 460, respectively. Once the prior distributions are estimated, predictions 466 can be generated for all segments for subject S using superset M_{S}. The predictions 466 can then be used to localize the sensor and determine whether the sensor is misplaced.

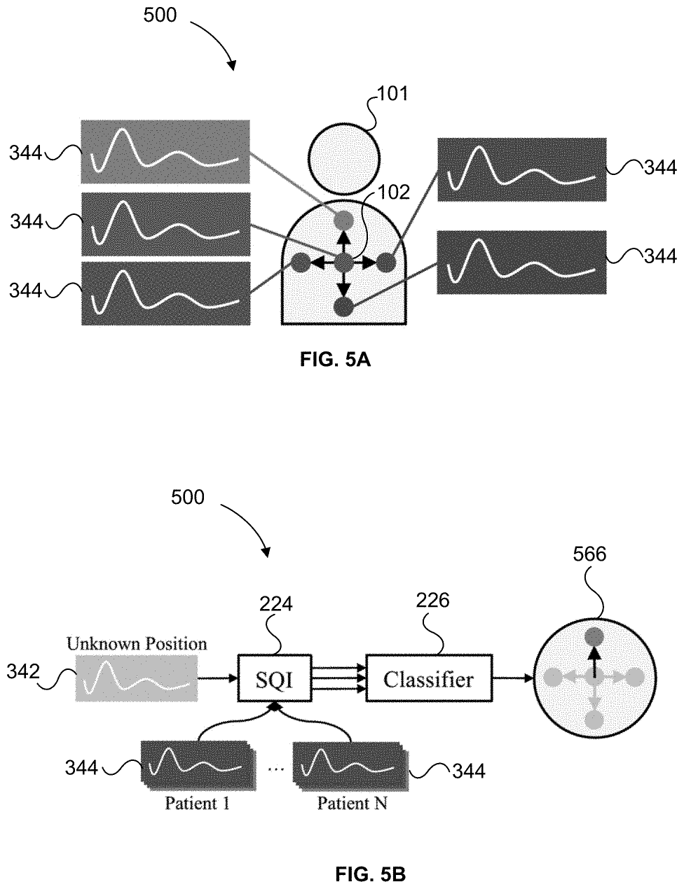

FIGS. 5A and 5B illustrate a method 500 of determining whether the motion sensor 102 is misplaced when mounted on a patient 101, in accordance with the disclosed technology. The disclosed technology can include generating templates 344 for each position of the motion sensor 102. As illustrated in FIG. 5A, a template 344 can be generated for signals obtained by the motion sensor 102 in various positions on the patient's 101 chest.

The templates 344 can then be used to determine a position of the motion sensor 102 on the patient's 101 chest when a signal 342 is obtained by the motion sensor 102. For example, and as will be described herein, a similarity between an obtained signal 342 and a template 344 can be computed to determine the most likely location of the motion sensor 102 on the patient's 101 chest.

As illustrated in FIG. 5B, similarity between a signal 342 and a template 344 can be computed using the SQI 224, including dynamic-time feature matching (DTFM), as previously described. To select an appropriate template 344, the disclosed technology can include defining X_{s,p} as a set of SCG signals for a subject (patient) s∈S and position p∈ [symbol] . For each signal 342 segment xᵢ∈X_{s,p}, the average distance dᵢ can be calculated by the following Equation (7).

$${d}_{i} = \frac{1}{M - 1} {\sum }_{j = 1}^{M} \{ j \neq i \} ( {x}_{i} , {x}_{j} ) \qquad Equation ( 7 )$$

where M is the number of signal 342 segments. The signals 342 can then be ranked based on their average distance, with lower distance representing greater fitness. The optimal template 344, or the template 344 that minimizes Equation (7), can then be selected. By selecting a template 344, or group of templates 344, that best fit the signal 342, the disclosed technology can more accurately analyze the signal 342 in comparison to the averaging-based template 344 generation previously described, and do so with less feature distortion.

Several classifiers 226 can be trained to map the feature vector of SQI 224 scores per segment to its motion sensor 102 position 566. The classifiers 226 can include linear (LDA) and quadratic discriminant analysis (QDA); k-nearest neighbors (k-NN) with 1, 10, and 100 neighbors; and support vector machines (SVM) with linear and quadratic kernels. These models can be chosen due to their relative simplicity in terms of hyperparameter tuning.

Once the motion sensor 102 position 566 is known, an output signal can be sent to a connected user interface (e.g., user interface 118) to indicate where the motion sensor 102 is located and whether the motion sensor 102 should be relocated. By selecting individual templates 344, or groups of templates 344, that best fit the signal 342, motion sensor 102 localization can more accurately be achieved without patient-specific calibration of the motion sensor 102.

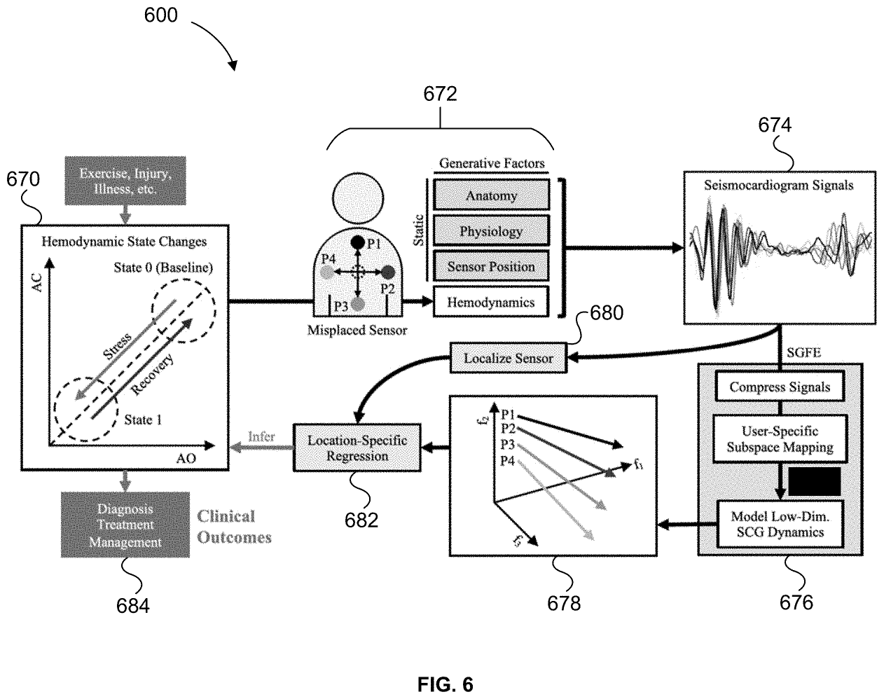

As previously described, the morphology SCG signals can be both patient-specific and highly transient which can make obtaining relevant information from the SCG signal difficult. To help overcome these issues, the disclosed technology can include a method 600, as illustrated in FIG. 6 and referred to as seismocardiogram generative factor encoding (SGFE), which models the SCG waveform as a stochastic sample from a low-dimensional subspace defined by a unified set of generative factors. In other words, by using SGFE, the SCG waveform can be modeled as a stochastic sample from an underlying (low-dimensional) dynamical system. Among other things, the method 600 can be used to enable algorithmic compensation for motion sensor 102 misplacement during generative factor inference to facilitate clinical outcomes 684.

For the sake of simplicity, a brief overview of the method 600 will first be described and then specific details about various elements of the method 600 will be described. As illustrated in FIG. 6, graph 670 depicts the consistent dynamics of the Aortic Opening (AO) and Aortic Closing (AC) during periods of stress (e.g., exercise, trauma, etc.) and recovery. While the particular trajectory in this state space in response to stress can be patient-specific, the dynamic behavior is largely preserved. Various generative factors 672 can affect the recorded SCG signal. Some of these generative factors 672 can be static (i.e., anatomy, physiology, and sensor position (e.g., motion sensor 102)) while other generative factors can be dynamic such as the hemodynamics (AO and AC) of the patient. The recorded SCG signal can be modeled as a stochastic sample 674 from these underlying generative factors 672. As will be described in greater detail herein, the method 600 can include using the SGFE to map 676 the SCG signals to a low-dimensional subspace. As illustrated in graph 678 the SCG signals can exhibit consistent dynamics in this learned low-dimensional subspace, however observed dynamics can be dependent on the position of the motion sensor 102. The position of the motion sensor 102 can be localized 680 using any of the previously described methods. The method 600 can further include applying position specific regression 682 to the learned subspace and the hemodynamic factors can be inferred independently from the other factors to facilitate clinical outcomes 684 for the patient.

For brevity in the following sections, shorthand will be used when describing matrices and vectors. Matrices, as described herein, are collections of row-wise vectors containing data from subsequent observations in the time interval T:=[1, T]. Consider an example T-by-M matrix of real numbers U∈R^{T′M}. It can be assumed that, U:=[u^{T}₁ . . . u^{T}_{T}]^{T} where uₜ∈R^{M}∀t∈T. In other words, U is composed of T vectors of length M, where each vector uₜ is an observation at time t. Since this notation is used frequently, the shorthand U:={u^{(M)}} is used. In any such matrix, u^{(i,j)} refers to the element of U in the i^{th} row and j^{th} column while u⁽ⁱ⁾ₜ refers to the i^{th} element in vector uₜ. Tuples, which are ordered sequences of objects, are indicated by lists of variables enclosed by parentheses. For example, the notation V:=(U, w) is used to define the variable Vas a tuple of the matrix U and vector w.

Since the SCG signal derives from the chest wall's response to underlying events, the signal can be abstracted as

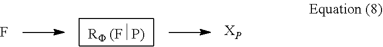

where F:={f^{(D)}}_{T} represents the hemodynamic generative factors of the signal 342, R is a response function that generates the waveform, and X_{P}:={x^{(M)}}_{T} is the set of observed SCG vectors from position P. The response function R is parameterized by Φ′, which represents the static generative factors 672 related to the patient's 101 anatomy and physiology, and is conditioned on the motion sensor 102 position P. Under the assumption that hemodynamic factors vary dynamically according to the state of the cardiovascular system, the factors at each timestep can be described as

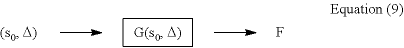

where s₀∈R^{K} is an initial state vector, A:={δ^{(L)}}_{T} represents changes in state at each point in the time period T, and G is a generator function that produces hemodynamic generative factors using this state information. Though the dimensionality of F and the state variables so and A are in reality unknown, acceptable values for D, K, and L in a computational model can be inferred, as will be described herein. The implications of modeling the SCG signal in this manner is that there can exist an encoder function E such that

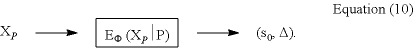

Given a set of observations X_{P} generated with Equation 8 the factors F that yielded these signals can be approximated by the system 100. Using Equations 9 and 10, this can be accomplished via

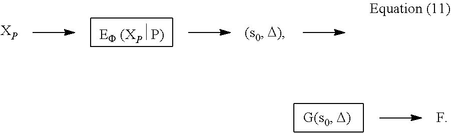

While the functions E and G are unknown, learning functions experimentally that approximate this behavior can allow inference of hemodynamic generative factors.

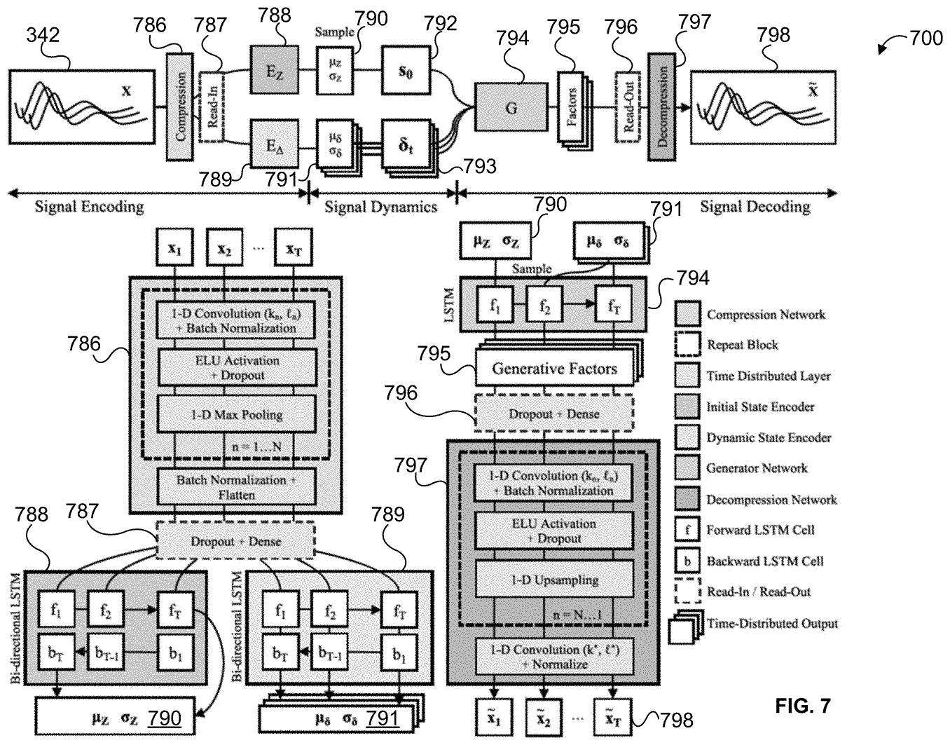

FIG. 7 illustrates a seismocardiogram generative factor encoder (SGFE) 700 according to the disclosed technology. The input to the SGFE 700 is a sequence X:={x^{(M)}} of T consecutive heartbeat-separated SCG signals 342 with length M.

To compress the signals, each signal xᵢ∈X is processed with the multi-layer convolutional network 786. This network 786 is composed of N=6 convolution blocks in series, which convolve the signal with each of kₙ filters (kernels) of length iₙ in the nth block with unit step. Convolutional networks 786 can be used in cardiovascular signal processing due to the temporal dependence of time-series data. The outputs of each convolution layer can be normalized before application of an exponential linear unit (ELU) activation function. Dropout regularization with a rate of 0.2 can be imposed on the output of the activation function. Dimensionality reduction can be induced by gradually decreasing the number of filters (kₙ=[64, 64, 32, 32, 16, 16]) and max pooling, which down-samples each signal by a factor of two. To accommodate for shorter signals, the kernel length can also be decreased (4=[7, 5, 5, 3, 3, 2]). The layers in this network are time-distributed, meaning the same operation can be performed for each signal xᵢ∈X.

Before modeling the dynamics present in X, the outputs of the compression network are flattened and passed through a dense “read-in” layer 787 with 64 input units, 2 (K+L) output units, and rectified linear unit (ReLU) activation with dropout regularization at a rate of 0.2. The read-in 787 and read-out 796 layers—also called mapping layers—exist because generative factors 795 can present differently as signal features across patients. Thus, though the subspace defined by the generative factors can be conserved, mapping into and out of this subspace can require compensation for signal heterogeneity by fitting these layers on a session-specific basis. In other words, the mapping layers capture anatomical and physiological differences—represented by Φ′ in Equations 8, 10, and 11—so that the dynamic model can focus on inferring factors that are common to the population.

Modeling dynamics requires estimation of the initial state so and change in state at each timestep A. As shown in FIG. 7, the former can be computed by the system 100 with a bi-directional long short-term memory (LS™) network 788 E_{Z}, where the output is the average between the final outputs of the forward and backward layers. The latter can also be computed by the system 100 with a bi-directional LS™ network 789 E_{Δ}, where an output δₜ is produced at each timestep as the average output between the forward and backward cells. However, since this can be a VAE instantiation, these values are not evaluated explicitly; rather, they are drawn from a Gaussian distribution, the parameters of which are explicitly evaluated. Thus, the output of E_{Z} is a tuple 790 (μ₀, σ₀), μ₀, σ₀∈R^{K}. The output of E_{Δ} at each timestep t∈T is a tuple 791 (μ_{δ,t} σ_{δ,t}), μ_{δ,t}∈R^{L}. The i^{th} element of the initial state vector s₀ is then sampled from

s₀⁽ⁱ⁾˜ [symbol] (μ₀⁽ⁱ⁾,σ₀⁽ⁱ⁾∀i∈[1,K]. Equation (12)

where N(μ,σ) is a Gaussian distribution with mean μ and standard deviation σ. Similarly, at each timestep t, the j^{th} element of the state change vector dt is sampled from

δₜ^{(j)}˜ [symbol] (μ_{δ,t}^{(j)},σ_{δ,t}^{(j)}∀j∈[1,L],t∈ [symbol] . Equation (12)

Note that each element in s₀ 792 and δₜ 793 is drawn independently. The probabilistic nature of the VAE yields a structured latent space, as nearby points will produce inherently similar outputs.

The generator network 794 can estimate the generative factors at each timestep based on the system state. The generator network 794 can be a uni-directional LS™ network, outputting a vector of factors fₜ∈R^{D} at each step t. As before, these factors 795 can be passed through a read-out 796 dense layer with D inputs and 64 outputs, which maps the generative factors 795 to corresponding signal features. Like the read-in layer 787, this mapping can be learned on a session-specific basis to account for changes in factor manifestation as signal features.

The translated factors can be used to construct the output signals X^{˜}:={x^{˜}^{_{(M)}}}_{T} with the decompression network 797. The decompression network 797 can be a mirror-image of the compression network 786, with the number and length of kernels applied in the reverse order and up-sampling by a factor of two rather than max pooling. The output of the decompression network 797 can be a convolution layer 798 with a single filter (k*=1) with length l*=l₁ such that the output is a single vector at each timestep. As will be appreciated by one of skill in the art, as more data is obtained from a patient, the data can be used to update the SGFE 700 model and infer generative factors 795 concurrently.

For visual analysis of subspace behavior, the goal of the following method can be to identify the pair of dimensions in the learned subspace F that encoded the most consistent linear trajectories. Linear trajectories can be expected to arise in the latent space because, as will be illustrated, AO and AC can be found experimentally to follow linear trends in exercise-recovery when plotted against one another.

To illustrate, for each session in a test set defined by the subject S∈[1, 10] and sensor (e.g., sensor 102) position P E {C, L, R, T, B}, the subspace mapping F∈R^{T×D} for each of N_{S,P} samples in a session can be concatenated to form the matrix F_{S,P}∈R^{T N S,P×D}. In this manner, each matrix F_{S,P} can contain the subspace encoding of all data for one of the sessions in the test set. These matrices can be further concatenated row-wise across all subjects to form the matrix F p for each sensor (e.g., motion sensor 102) position. Thus, F_{P} can contain the subspace encoding of all data from sessions from a particular sensor position.

For illustrative purposes, the following can then be performed for all P. For each pair of column vectors (fᵢ, fⱼ)∈F_{P}, i≠j, linear regression can be used to find the optimal linear fit between fᵢ and fⱼ. The pair i, j in which the coefficient of determination (R²) of the linear fit averaged across all P was maximal can be selected as the optimal axis pair for further analysis. Subspace trajectories can be visualized by plotting the resultant vectors f₁ and f₂ against one another.

A second qualitative analysis can be performed to determine whether the identified dimensions can contain useful information about the known generative factors AO and AC. An ICG-derived rAO interval can be plotted against the rAC interval on a scatter plot. Best-fit lines can then be overlaid on data from each subject to better visualize the trajectories of these intervals. For the same subjects, the subspace mappings f₁ and f₂ from the same session for the central sensor location can be plotted on a scatter plot. Best-fit lines can be overlaid on the subspace encoding for each patient in order to observe whether changes in rAO/rAC trajectories can be reflected by the identified dimensions.

Though the hyperplane defined by f₁ and f₂ can be a suitable subspace in which to observe the consistent dynamics of SCG signals, it can be sub-optimal for visualizing the effects of changing motion sensor 102 state on observed dynamics. To do so more effectively, principal component analysis (PCA) can be used to find an informative three-dimensional representation of the subspace F, and the average trajectory for each motion sensor 102 position can be plotted in these PCA dimensions for visualization.

To do so, the matrix F p can be concatenated across positions by the system 100 to form Fₜₒₜ∈R^{T Ntot×D} where Nₜₒₜ is the total number of samples in the testing set. The matrix Fₜₒₜ thus can contain the subspace mappings for all samples in the testing set. PCA can then be performed on Fₜₒₜ to obtain the transformation A∈R^{D×D} mapping dimensions of Fₜₒₜ into the orthogonal subspace defined by PCA dimensions.

The following can then be performed by the system 100 for each matrix F_{S,P}, which can contain the subspace encoding for the session with subject S and position P. Each of the 10 matrices F_{S,P}, S∈[1, 10] can be averaged elementwise to obtain a session-averaged matrix F_{P}. F_{P} can thereby contain the subspace encoding for position P averaged across all subjects. Subsequently, this matrix can be transformed using the matrix A to obtain A_{P}=F_{P} A, the mapping of F_{P} in the PCA subspace. Finally, for each position, the first three dimensions of A_{P} can then be plotted on a scatter plot for visualization (e.g., output to the user interface 118).

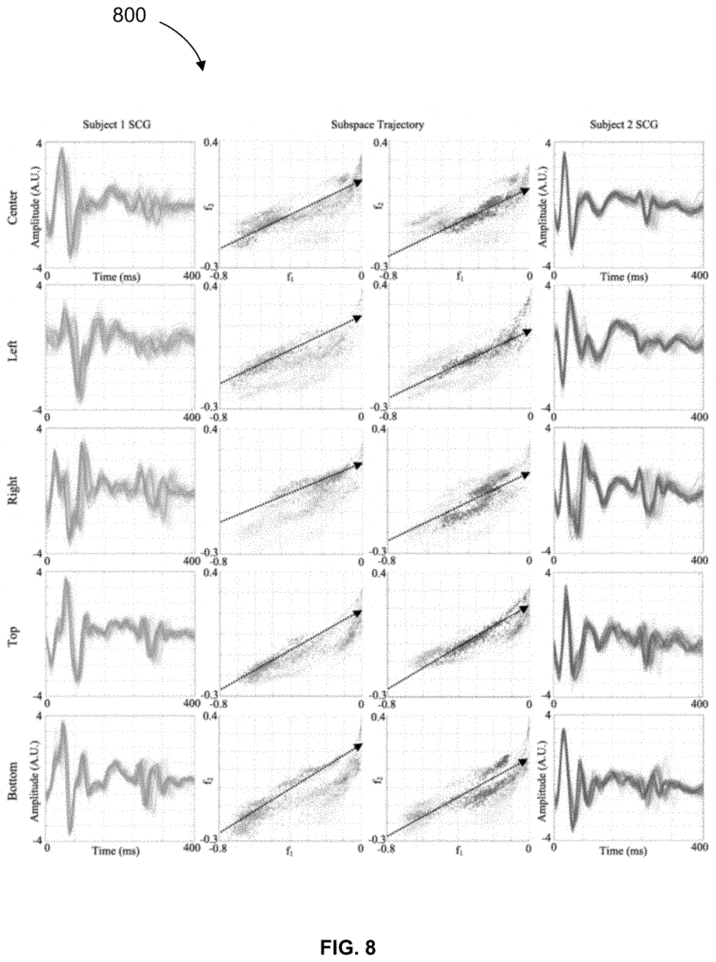

FIG. 8 depicts subspace mappings 800 of recovery-period SCG data for two example patients 101. The rows of the FIG. 8 represent each of the five different sensor positions previously described (e.g., C, L, R, T, B). From the first and last columns of FIG. 8, it is apparent that signal morphology between the patients—and even at different sensor locations for the same patient—can vart substantially. This time-domain variability is juxtaposed with trajectories in the learned subspace which are largely conserved. Specifically, the subspace mapping of the signal during this period can follow an approximately linear trajectory in the reference frame defined by the selected subspace dimensions f₁ and f₂.

The left and right columns of FIG. 8 show a subset of the amplitude-normalized SCG data from patients 1 and 2 respectively, with the second and third columns showing the corresponding subspace trajectories respectively. The axes represent learned subspace dimensions f₁ and f₂. The small gray points in the figure represent subspace mappings with the same sensor position from the remaining patients in the testing set. Trajectory directions are overlaid (black, dotted). The indication A.U. represents arbitrary units.

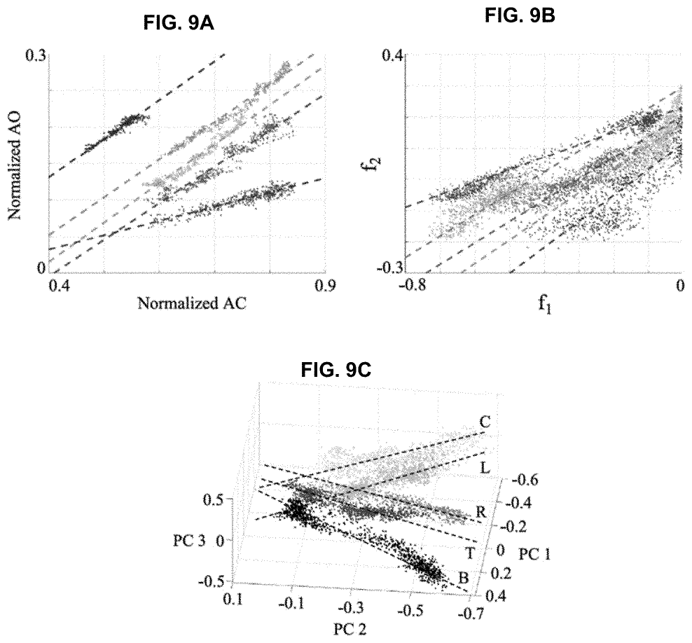

FIG. 9A represents ICG-derived AO and AC points during exercise-recovery for five example patients in a test set. AO and AC are shown as scatter points while best-fit lines for the scatter points are overlaid as dashed lines. FIG. 9B illustrates subspace trajectories in dimensions f₁ and f₂ from centrally-placed sensors (e.g., motion sensor 102) for the same patients. Subspace mappings are similarly shown as scatter points with best-fit lines overlaid as dashed lines. FIG. 9C illustrates trajectories in PCA dimensions of F for SCG signals from each of the five motion sensor 102 positions averaged across all patients. The positions include center, left, right, top, and bottom. The trajectories are also indicated with black dashed lines. As will be appreciated by one of skill in the art, the disclosed technology can enable position-specific mapping to infer hemodynamic variables as illustrated in FIG. 9C.

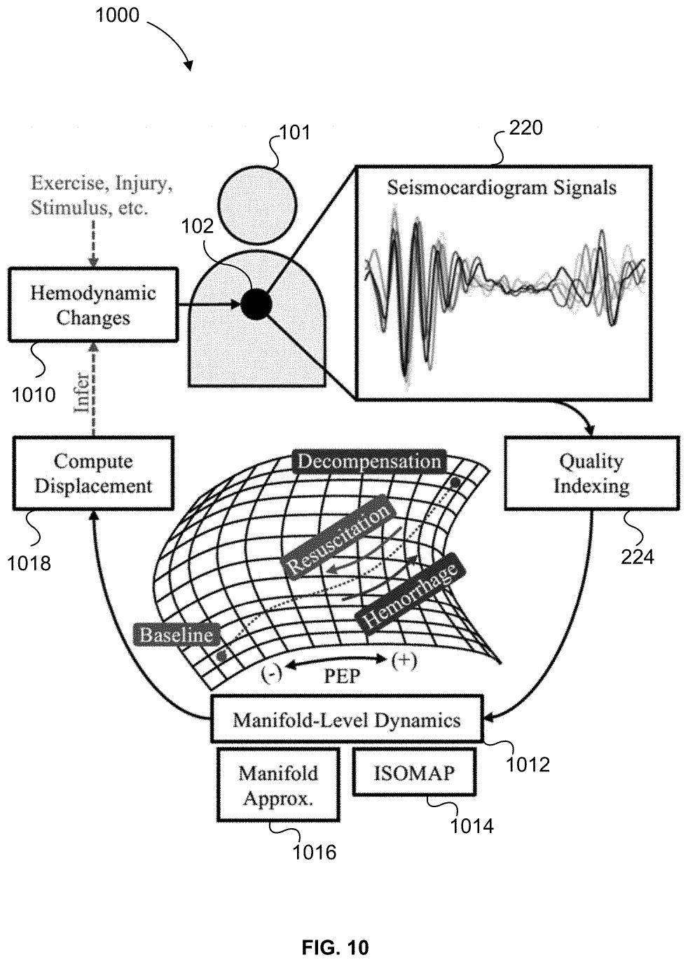

As illustrated in FIG. 10, the disclosed technology can include a method 1000 of determining physiological changes during hemorrhage and subsequent fluid resuscitation via PEP estimation. As will be described in greater detail herein, the method 1000 can be used to detect and analyze low-dimensional manifold structures of SCG signals during periods of hemodynamic change. Displacement along the manifold can be linearly-related to changes in PEP and used to determine a hemodynamic change of the patient's 101 body.

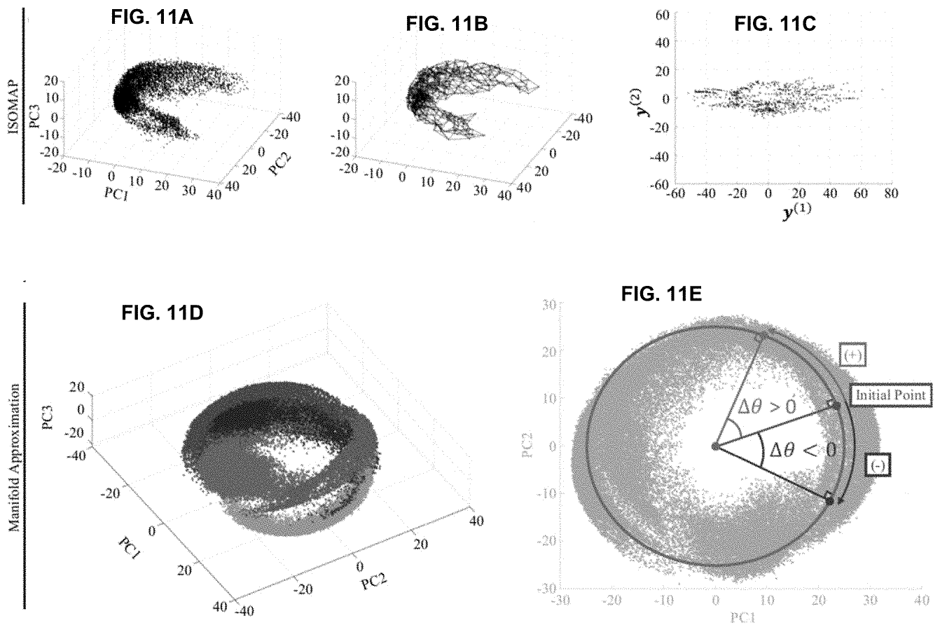

The motion sensor 102 can obtain SCG signals 220 when mounted to a patient's 101 chest during periods of hemodynamic change 1010. The SCG signals 220 can be processed by the SQI 224 previously described (illustrated as quality indexing in FIG. 10) to remove low-quality signals. The SCG signals 220 can be compared to a manifold structure inherent to SCG signals 220 by non-linearly mapping the SCG signals 220 to positions along the low-dimensional manifold 1012 to infer hemodynamic changes. Linear dimensionality reduction with PCA can be used to visualize the manifold structure inherent to SCG signals 220. As will be described in greater detail herein, nonlinear dimensionality reduction, such as either an ISOMAP 1014 or a manifold approximation 1016, can be used to analyze the distance traveled along manifold to infer hemodynamic changes. For example, a computation of the displacement 1018 along the manifold can be used to determine (or infer) the hemodynamic changes 1010 of the patient 101. As will be appreciated by one of skill in the art, the disclosed technology can be used to non-invasively detect potentially harmful or dangerous cardiac or hemodynamic changes of the patient 101. For example, as described further herein, the disclosed technology can be used to create a graph that can be used to detect hemodynamic changes such as blood loss from a hemorrhage as illustrated in FIGS. 11A-11E. For this reason, the disclosed technology can be used for reliable physiological estimation from SCG signals during trauma-induced hemorrhage and subsequent treatment. Namely, estimating indicators of cardiomechanical function such as PEP noninvasively can enable new clinical tools to allow healthcare providers to manage trauma injury, serving as additional indicators of the severity of hemorrhage and the patients' response to fluid resuscitation.

The disclosed technology can include using an ISOMAP 1014 algorithm to learn and re-embed the manifold. As illustrated in FIGS. 11A-11C, a graph can be constructed from a sub-sampling of points Gᵢ={g₁, g₂, . . . , g_{L}} from the overall dataset Xᵢ={x₁, x₂, . . . , x_{M}}, which form the nodes of the graph. A connection between nodes gⱼ and gₖ can be formed if and only if there exists a point x₁ in the original dataset whose nearest neighbors are gⱼ and gₖ as per the Euclidian distance. The geodesic distance between each pair of nodes gⱼ, gₖ∈Gi can then estimated by computing the shortest path between each pair of nodes that traverses the graph's connections. This can be performed, for example, by the Floyd-Warshall algorithm.

The manifold can then be re-embedded by the system 100 to learn a mapping f:R^{L×N}→Y from the observation space of Gᵢ to a lower-dimensional space Y=R^{L×D} which preserves the geodesic distances between pairs of points in the graph. As an example, “classical” multidimensional scaling (MDS) to learn this mapping. Specifically, MDS can minimize the loss function

$$L ( f , {G}_{i} ) = {( \frac{{\sum }_{j , k} {( {d}_{Y}^{jk} - {d}_{G}^{jk} )}^{2}}{{\sum }_{j , k} {( {d}_{Y}^{jk} )}^{2}} )}^{1 / 2} , \qquad Equation ( 14 )$$

where d^{jk}_{Y}=∥f (gⱼ)−f(gₖ)∥₂ is the Euclidian distance (or the l₂-norm) between f(gⱼ) and f (gₖ) in the output space Y and d^{jk}_{G} is the estimated Euclidian distance between the feature vectors gⱼ and gₖ.

To obtain an accurate embedding of SCG manifolds the SQI 224 can first be applied by the system 100 to the SCG data to remove outliers. As the final step of processing, the latent variable Δy can be obtained by the system 100 for each patient by computing the offset of each element in yᵢ⁽¹⁾ from the initial element in the vector yᵢ⁽¹⁾ (0), such that Δyᵢ=yᵢ⁽¹⁾−yᵢ⁽¹⁾ (0). This can be done in order to obtain the displacement 1018 of each point on the manifold rather than the absolute position. This latent variable can be used to estimate the change in PEP (APEP) via linear regression.

Alternatively, or in addition, manifold approximation 1016 can be used by the system 100 to learn and re-embed the manifold as illustrated in FIGS. 11D-11E. For each patient 101, a separate PCA transformation can be learned from the data obtained from other patients 101 (Xi⁻) and applied to the data from the held-out patient (Xi). The initial sample from the patient 101 can then be mapped to the nearest point on a unit circle in the plane of PC1 and PC2, centered at the origin. Each subsequent point can then also be mapped to the nearest point on the unit circle, and the angular offset between the new point and the initial point can be recorded. This can result in a vector Δθᵢ containing the angular offset for each SCG signal in Xᵢ. This process can also be repeated with an SQI cutoff increasing from 0% to 20% in increments of 5%. The latent variable Δθ can then be used to estimate APEP in an analogous manner to Δy from ISOMAP 1014. Unlike the ISOMAP 1014 algorithm, the manifold approximation 1016 algorithm has O_{(N)}, and is thereby much more rapid.

As will be appreciated, the methods described herein can be varied in accordance with the various elements and implementations described herein. That is, methods in accordance with the disclosed technology can include all or some of the steps described above and/or can include additional steps not expressly disclosed above. Further, methods in accordance with the disclosed technology can include some, but not all, of a particular step described above. Further still, various methods described herein can be combined in full or in part. That is, methods in accordance with the disclosed technology can include at least some elements or steps of a first method and at least some elements or steps of a second method.

While the present disclosure has been described in connection with a plurality of example aspects, as illustrated in the various figures and discussed above, it is understood that other similar aspects can be used, or modifications and additions can be made to the described subject matter for performing the same function of the present disclosure without deviating therefrom. In this disclosure, methods and compositions were described according to aspects of the presently disclosed subject matter. But other equivalent methods or compositions to these described aspects are also contemplated by the teachings herein. Therefore, the present disclosure should not be limited to any single aspect, but rather construed in breadth and scope in accordance with the appended claims. Moreover, various aspects of the disclosed technology have been described herein as relating to methods, systems, devices, and/or non-transitory, computer-readable medium storing instructions. However, it is to be understood that the disclosed technology is not necessarily limited to the examples and embodiments expressly described herein. That is, certain aspects of a described system can be included in the methods described herein, various aspects of a described method can be included in a system described herein, and the like.

What is claimed is:

1. A method of detecting morphological changes using a wearable cardiac monitor, the method comprising:
receiving seismocardiographic data from a motion sensor;
determining, based at least in part on the seismocardiographic data, a classifier of the seismocardiographic data;
determining, based at least in part on the seismocardiographic data, a signal quality index of the seismocardiographic data;
determining, based on the classifier and the signal quality index, a quality of the seismocardiographic data; and
outputting, to a graphical user interface, the quality of the seismocardiographic data.

2. The method of claim 1, wherein the classifier comprises multiple classifiers, and wherein the method further comprises:
performing ensemble prediction by utilizing the multiple classifiers to create an ensemble classifier.

3. The method of claim 1 further comprising:
retrieving a reference template, the reference template comprising representative seismocardiographic data; and
determining the signal quality index by determining a distance between the seismocardiographic data and the reference template.

4. The method of claim 3 further comprising:
estimating the distance between the seismocardiographic data and the reference template by dynamic-time feature matching, wherein dynamic-time feature matching comprises:
determining a local minimum and a local maximum of the seismocardiographic data;
determining a local minimum and a local maximum of the reference template;
determining a prohibited intersection point;
determining a candidate point; and
generating a warp path through the candidate point.

5. The method of claim 1 further comprising:
retrieving a location-specific reference template, the location-specific reference template comprising representative seismocardiographic data at a predetermine location; and
determining a location of the motion sensor by at least determining a distance between the seismocardiographic data and the location-specific reference template.

6. The method of claim 5 further comprising:
performing a seismocardiogram generative factor encoding;
comparing the seismocardiographic data to a low-dimensional subspace; and
applying a position-specific regression to the low-dimensional subspace to determine a hemodynamic factor.

7. The method of claim 5 further comprising:
comparing the seismocardiographic data to a low-dimensional manifold;
performing a manifold approximation to analyze a distance traveled along the low-dimensional manifold; and
determining a hemodynamic change based on the distance traveled along the low-dimensional manifold.

8. A wearable cardiac monitoring system comprising:
a motion sensor configured to detect movement of a user's chest and output seismocardiographic data; and
a non-transitory, computer-readable medium having instructions stored thereon that, when executed by one or more processors, cause a controller to:
receive the seismocardiographic data from the motion sensor;
determine, based at least in part on the seismocardiographic data, a classifier of the seismocardiographic data;
determine, based at least in part on the seismocardiographic data, a signal quality index of the seismocardiographic data;
determine, based on the classifier and the signal quality index, a quality of the seismocardiographic data; and
output, to a graphical user interface, an indication of the quality of the seismocardiographic data.

9. The wearable cardiac monitoring system of claim 8, wherein the classifier comprises a property of a source distribution of the seismocardiographic data.

10. The wearable cardiac monitoring system of claim 8, wherein the classifier comprises multiple classifiers, and
wherein the instructions, when executed by the one or more processors, further cause the controller to:
perform ensemble prediction by utilizing the multiple classifiers to create an ensemble classifier.

11. The wearable cardiac monitoring system of claim 8, wherein the instructions, when executed by the one or more processors, further cause the controller to:
retrieve a reference template, the reference template comprising representative seismocardiographic data; and
determine the signal quality index by determining a distance between the seismocardiographic data and the reference template.

12. The wearable cardiac monitoring system of claim 11, wherein the instructions, when executed by the one or more processors, further cause the controller to:
estimate the distance between the seismocardiographic data and the reference template by dynamic-time feature matching.

13. The wearable cardiac monitoring system of claim 12, wherein dynamic-time feature matching comprises:
determining a local minimum and a local maximum of the seismocardiographic data;
determining a local minimum and a local maximum of the reference template;
determining a prohibited intersection point;
determining a candidate point; and
generating a warp path through the candidate point.

14. The wearable cardiac monitoring system of claim 8, wherein the instructions, when executed by the one or more processors, further cause the controller to:
retrieve a location-specific reference template, the location-specific reference template comprising representative seismocardiographic data at a predetermine location; and
determine a location of the motion sensor by at least determining a distance between the seismocardiographic data and the location-specific reference template.

15. The wearable cardiac monitoring system of claim 14, wherein the instructions, when executed by the one or more processors, further cause the controller to:
perform a seismocardiogram generative factor encoding;
compare the seismocardiographic data to a low-dimensional subspace; and
apply a position-specific regression to the low-dimensional subspace to determine a hemodynamic factor.

16. The wearable cardiac monitoring system of claim 14, wherein the instructions, when executed by the one or more processors, further cause the controller to:
compare the seismocardiographic data to a low-dimensional manifold;
perform a manifold approximation to analyze a distance traveled along the low-dimensional manifold; and
determine a hemodynamic change based on the distance traveled along the low-dimensional manifold.

17. The wearable cardiac monitoring system of claim 8, wherein the controller is configured to wirelessly receive the seismocardiographic data from the motion sensor.

18. The wearable cardiac monitoring system of claim 17, wherein the controller is a remote server configured to wirelessly receive the seismocardiographic data from the motion sensor.

19. The wearable cardiac monitoring system of claim 8, wherein the controller is integrated with the motion sensor into a single wearable device.

20. The wearable cardiac monitoring system of claim 8, wherein the instructions, when executed by the one or more processors, further cause the controller to:
receive a first input to change a threshold value of the classifier; and
receive a second input to change a threshold value of the signal quality index.
